(19) United States
(12) Patent Application Publication
(10) Pub. No.: US 2011/0147986 A1
(43) Pub. Date: Jun. 23, 2011

(54) Oxygen Resistant Organic Peroxides for Reticulation/Cure and Their Use in the Process of Continuous Vulcanization in Hot Air Tunnel

(76) Inventor: Antonio D'Angelo, Santana do Parnaiba-Sao Paulo (BR)

(21) Appl. No.: 13/060,021

(22) PCT Filed: Aug. 21, 2008
(86) PCT No.: PCT/IB08/53363

§ 371 Date: Feb. 21, 2011

(51) Int. Cl. B29C 71/02; C08L 23/16; C07C 409/16; C07C 409/38; C07C 409/32

(52) U.S. Cl. 264/236; 524/570; 568/558; 568/561; 560/302; 568/566

(57) ABSTRACT

This application refers to Organic Peroxides of the Dialcyl, Perester, Perketal, and Dialkyl classes, which were modified to resist to oxygen, destined to be used in the process of production of Power Wires and Cables, Shapes, flocculated, compact and spongeous, used for the assembly of sealing of doors, trunks and windows in the automobile industry and civil construction, and also in the process of continuous vulcanization in hot air tunnel, in presence of oxygen, with the application of modified Organic Peroxides.

### INTRODUCTION

This application refers to organic peroxides of Dialcyl, Peresther, Perketal, and Dialkyl Classes that have been modified to become oxygen resistant, destined to be used in the process of production of Power Wire and Cables, and also Shapes, flocculated, compact and spongeous, used for the assembly of sealing of doors, trunks and windows in the automobile industry and civil construction, as well as in the process of Continuous Vulcanization in Hot Air Tunnel, in the presence of oxygen, with the application of modified organic peroxides.

This invention was developed along a decade of industry and laboratory research and development and is based upon five (5) basic and fundamental aspects;
1—) Research and development of Specially Modified Organic Peroxides of the chemical classes; Dialcyl, Peresters, Perketals, and Dialkyl, which would be resistant to molecular oxygen present in the continuous vulcanization process in hot air tunnel, since the conventional Organic Peroxides of the same chemical classes do not resist because they undergo cleavage (the surface becomes tacky)

2—) The adaptation referring to the best components that could be used in formulations was researched, from the most varied types of polymers, saturated or unsaturated and their blends, as well as oils, process auxiliaries, desiccants, antioxidants, and the most appropriate modified Organic Peroxides as a function of the process temperatures.

3—) Various types of extruders were tested and checked, in relation to their length, extrusion speed, temperatures and cooling.

4—) The matrixes used in the different types and thickness of shapes were also studied.

5—) A wide study was conducted together with industrial verification regarding the improvement and optimization of the various processes for continuous vulcanization in hot air tunnel, in relation to speed, temperatures, heating zones, mixtures of cure systems such as using Microwave together with the process of continuous vulcanization in hot air tunnel.

After all these adjustments, which we shall name as the 5 stages of design development, we arrived at the purpose of this patent application since we obtained the desired and inedited absence of cleavage or non tacky surface in the vulcanized compound, with the described process, attesting the efficacy of the Modified Organic Peroxides, resistant to oxygen.

The success obtained in all the industrial tests was assured by the interaction of the compound formulation with the acceleration obtained with these new modified Organic Peroxides, resistant to oxygen.

The perfect adjustment of the formulation was fundamental, the adjustment of the continuous vulcanization process, in hot air tunnel, in presence of oxygen, since this allowed us to overcome the unfeasibility concept of the peroxide cure in presence of molecular oxygen.

This technology replaces, with advantages, the conventional vulcanization system with sulfur and accelerators used until now in the continuous vulcanization process in hot air tunnel, in presence of oxygen.

### BACKGROUND OF THE INVENTION

The compounds that are formulated and structured based upon various polymers and their blends, listed below, were accelerated with these new modified Organic Peroxides, resistant to oxygen, which are donors of free radicals, and then they were extruded in extruders of various lengths (LD from 1 to 22 meters), vulcanized by the process of continuous vulcanization in hot air tunnel, in presence of oxygen, in various types and sizes of vulcanization tunnels (from 12 to 50 meters long) and at various vulcanization temperatures from 120° C. to 400° C., in tunnel heated with thermal oil or electric resistances, where occurs the reticulation/cure of the compound (bonding or crosslinking).

### Reticulation/Cure or Linking Via Peroxides

### C—R—C or Modified C—C

Replaces the conventional vulcanization/cure via sulfur and accelerators due to having stronger bonding force;

### Conventional Vulcanization

### C—S or C-s-C

Molecular oxygen was for a long time considered as harming the peroxide cure, except in the system used for continuous vulcanization in hot air tunnel, for silicone base polymer, when using peroxide based upon dichlorobenzoyl at 50%.

This invention describes the vulcanization process and the specially modified Organic Peroxides, with different additives, for resisting to oxygen, in order to avoid cleavage in the various rubber compounds vulcanized in hot air tunnel.

This technological Innovation has been developed for over a decade and may be applied in the industries for transforming rubber and plastic, in the reticulation/cure of various polymers; EPDM—Ethylene Propylene Diene (thermal polymer) EPM—Ethylene Propylene (Copolymer), NBR—Butadiene Acrylonitrile, SBR—Styrene Butadiene, SSBR—Styrene Butadiene (per solution) CPE—Chlorinated Polyethylene, CSM—Chlorosulfonated Polyethylene, CR—Polychloroprene (Neoprene), E.V.A—Ethylene Vinyl Acetate, LDPE—Low Density Polyethylene, HDPE—High Density Polyethylene, POE—Polyethylene Octene (Engage) and their blends.

The invention of these modified Organic Peroxides and the improvement of the continuous vulcanization process in hot air tunnel allows the inedited use of these technologies for the manufacture of the following products; POWER WIRES AND CABLES, SHAPES FOR SEALING, HOSES, PIPES, BORDERS, etc., based upon various saturated and unsaturated polymers, as already mentioned, and their blends, without occurring the tacky surface phenomenon or cleavage in the surface of the products.

This innovative technique, as compared with the traditional systems based upon cure with sulfur and accelerators, provides as advantages the final features of the products, such as: improved mechanical properties, significant improvement in the permanent deformation under compression and particularly, thermal aging.

### PRIOR ART

There are patents that use various compounds that physically cover the surface of vulcanizing elastomer in order to avoid the action of oxygen.

E.g., the U.S. Pat. No. 4,439,388 describing the use of boric acid as a treatment before the cure with hot air. This covering technique is laborious, since it has to be removed and disposed off after the conclusion of the crosslinking reaction.

The U.S. Pat. No. 4,983,685 describes the use of accelerator compounds selected among the following classes: (a) imidazol compounds, (b) compounds based upon thiourea, (c) thiazole compounds, (d) thiouran compounds, (e) dithiocarbonate compounds, (f) phenolic compounds, (g) triazole compounds, and (h) amine compounds, which are accelerators for vulcanization with sulfur with optional presence of antioxidants, antidegrading compounds and similar used for elastomer vulcanization for reducing the action of oxygen on the surface of the compounds vulcanized with peroxides.

Among the optional ingredients suggested for inclusion in the formulations for increasing crosslinking we have the N,N′-M-Phenylene bismaleimide.

There is no mention that this bismaleimide compound could provide a considerable effect in the stability of certain compounds by reducing the tackiness during the cure with peroxides, which provide free radicals during decomposition in the presence of molecular oxygen.

The use of various accelerators and sulfur, particularly with peroxide, provides a reduction of tacky surfaces of the polymers cured with peroxide, however the physical properties are reduced, mainly the most required by the industry that is DPC or compression set.

The U.S. Pat. No. 4,983,685 does not mention that the elastomers of silicone bismaleimides and biscitraconimides of this invention used in combinations with antiozonizing before conducting the vulcanization with sulfur and antioxidants and/or polysulfide polymers in cures with free radicals without tacky surfaces and improved physical properties could happen as a result of the mentioned materials, and this effect happens only in particular cases.

The U.S. Pat. No. 4,334,043 presents the use of surface treatment of the polymer compounds with metal salts, organic, inorganic or lantanides.

This patent informs the absence of tacky surfaces in the compounds cured with conventional peroxides. Other means for controlling cleavage are not mentioned, except the previously known techniques that eliminate the contact with air in the rubber surface.

Some authors declare that using elemental sulfur has a negative effect on the final physical properties of the cured elastomers, from the point of view of cure with sulfur, when compared with peroxide cure.

The U.S. Pat. No. 4,575,552 claims that using specific combinations of phenolic antioxidants, metal salts of diethylcarbamates and m-phenylene-dimelaimide provide a polymer vulcanized with peroxide with thermal and hydrolytic stability for geothermal applications, but there is no mention to the presence of hot air and inhibition of surface tackiness.

The patent application No. US 20040180985 reports the absence of tackiness in silicone base polymer, cured with Organic Peroxides, in presence of air, but this is not presented as a solution or technology for continuous vulcanization process in hot air tunnel, in presence of oxygen.

### RELEVANT PUBLICATIONS

As follows are listed some references;
Continuous Vulcanizing Systems for Rubber and XLPE Cables
Compounding for continuous Curing—By M. A. Schoen Beck—Akron Rubber
Compounding elastomers for continuous curing—Rubber World April 1983
Continuous Vulcanization of Rubber—by Ven L. Lue
The continuous Vulcanizing Extruded articles continuously—Bayer do Brasil

None of the references informed herein, isolated or in combination, suggests the solution required by the applicant, i.e.; the innovation of the technology of modified Organic Peroxides, resistant to oxygen, and their inedited application to continuous vulcanization process in hot air tunnel, in presence de oxygen, without occurring the cleavage phenomenon on the surface of the reticulated/cures products.

### BRIEF DESCRIPTION OF THE DRAWINGS

FIG. 1 shows a flow diagram for validation of the technology for continuous vulcanization process in hot air tunnel, in presence of oxygen.

FIG. 2 shows a schematic process and obtainment of test samples.

FIG. 3 shows a block flow diagram of the process in hot air tunnel.

### DETAILED DESCRIPTION OF THE INVENTION

The research and development for this invention started with the attempt to use conventional Organic Peroxides, of dialkyl, dialkyl, peresther and perketal classes, donors of free radicals, for acceleration of compounds based upon EPDM—ethylene propylene diene (third monomer), the most used polymer for extruded compounds for production of compact and sponge shapes, manufactured by continuous vulcanization process in hot air tunnel; in presence of oxygen, as a replacement for the conventional cure system based upon sulfur and accelerators, but which did not result in a well vulcanized part, since good physical characteristics were not obtained and due to the occurrence of the cleavage phenomenon (tacky surface of the product).

As from this point, we started to study the chemical reactions, obtained from modifications in the types of conventional Organic Peroxides, of Dialkyl, Dialcyl, Peresther and Perketal classes, with various additives, aiming for increased resistance to molecular oxygen, and for more efficient action as a source for donating free radicals to the polymer chain, and improved resistance to tacky surface on the products, with better performance in the continuous vulcanization in hot air tunnel, in presence of oxygen, with the introduction of other bonding radicals in the molecular chain of the various described polymers; C—R—C type.

### FUNCTIONING OF THE INVENTION

Various compounds were produced to validate this invention, based upon the various polymers, already mentioned, the defined ingredients, and these compounds were weighed, mixed in closed mixer, Banbury type, accelerated, in open mixer of two roll calendar type, with the various modified Organic Peroxides, resistant to oxygen.

In the process sequence, the compounds were extruded, in various types of extruders, with various diameters, process temperature, length, matrixes and speed, for production of the pre-cast, after this and according to the matrix, the products were vulcanized by the continuous vulcanization process in hot air tunnel, in presence of oxygen.

The used temperatures varied as a function of the type of used peroxide, allowing to obtain an excellent grade and status for the reticulation/cure, without the occurrence of tacky external surface on the shape, verifying that the final properties complied with and were in accordance with the market standards and specifications, according to comparative tests to be described in this document.

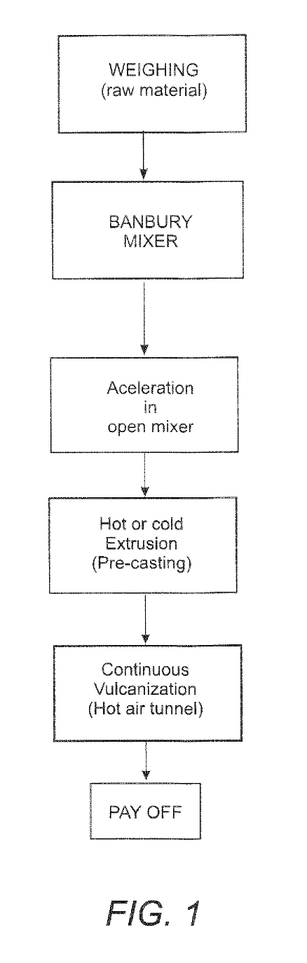

Based upon FIG. 1, we describe as follows the process stages for obtaining flocculated, compact and spongeous shapes with the continuous vulcanization process in hot air tunnel, in presence of oxygen.

E.1—Weighing

The weighing of the raw-material is conducted in a room dedicated for this purpose, thus avoiding product contamination. This is the first stage of the process that shall be conducted in the correct sequence according to the production sheet and with accuracy, since any fault could interfere in the final result of the rubber product.

E.2—Mix Processing in Banbury

Basically, the Banbury is a closed mixer, consisting of a chamber whose walls are controlled with a complex system of gears and electric motors. A hydraulic plunger is located inside the throat of the machine, for maintaining the mix under pressure, filling all the empty spaces inside the chamber, which aid with the good dispersion of the ingredients and elastomers.

The mass is crushed under the shearing action of the rotors against the chamber walls, being dragged to the space between the two rotors, because the rotors turn at different speeds (friction).

The already mentioned polymer base compounds, but mainly based upon EPDM—Ethylene Propylene Diene, are processed in the Banbury, particularly when the formula/recipe contains high contents of charges and plasticizers.

For short mix cycles, the upside-down system is the most commonly used.

The upside-down system consists in feeding the Banbury (empty) with the charges, plasticizers, process aids and activators, lowering the mortar and mixing for approximately 1 minute, and then lifting the mortar, adding the polymer, lowering the mortar and processing the mix for 4 to 5 minutes.

If the equipment allows perfect temperature control, and the compound has good processing safety, the Modified Organic Peroxides, known as vulcanization agents, may be added directly to the Banbury at temperatures up to 150° C., since this technology already includes the modified Organic Peroxides with larger scorch safety.

If the temperature of the Banbury is over 150° C., the acceleration of the mass shall be conducted with open mixers, calendars or cylinders with 2 rolls, at temperatures between 90 and 150° C. in order to avoid the occurrence of pre-cure of the mass.

E.3—Processing of Acceleration in Open Mixer (Cylinder)

Historically, the cylinder (open mixer) was the first mixing machine used in the rubber industry, basically consisting of two steel cylinders, very hard, horizontally arranged, turning in opposite directions, with different peripheral speeds, over which are placed the rubber and the ingredients to be mixed, often being used as a substitute for the closed mixers, however the largest use is for: homogenization, pre-heating, milling and acceleration.

E.4—Conformation of Products of Saturated or Unsaturated Polymers.

The development of new technologies for the production of rubber products resulted in the need for adapting the conformation of compounds in order to comply with the production demand.

Among the new technologies we may mention using peripheral controllers for extrusion.

The extruders are machines that press the elastomeric compound thru a hole or matrix producing a strip of material with a determined shape, the matrix has various sizes for better adaptation to the required application.

The screw extruder is most commonly used and includes a feeding spout, a screw that operates in a cylindrical body with a jacket for water circulation, a head and a matrix for producing the pre-conformed mass already with the definitive shape.

The screw is driven by an electric motor with reduction gears, and pushes the compound thru the cylinder inside the head, generating a pressure that is relived by passing the material thru the matrix, forming the desired shape. This type of extruder is destined to continuous operation and may have manual or automatic feeding.

The pre-formation of the product consists in pre-casting the compound before the cure, i.e., giving a previous shape to the product before the final molding. This process aims for maximum approximation of the volume of the compound to be molded with the volume of the product, thus avoiding wasting the compound and maintaining the dimensional constancy of the product.

E.5—Continuous Vulcanization System (Hot Air Tunnel).

The continuous vulcanization of a rubber compound is not a particularly new process, since it already has been used for a long time in the wire and cable industry, there are five continuous vulcanization systems that are more commonly currently used:
Hot air tunnel,
Vulcanization in Steam Tube.
Liquid Medium for Cure,
Fluidized Bed
High Frequency Microwave

Notice that none of these systems use a mold, thus the product should be formed and remain dimensionally stable before the cure.

With the exception of the system of vulcanization in steam tube, all the systems work at atmospheric pressure.

The innovative technology of modified Organic Peroxides was specially developed for using the hot air tunnel process, which essentially is an open furnace (tunnel) where hot air is circulated.

This tunnel is lined with a jacket that heats the inner air by transporting heat with the thermal oil that circulates in the jacket, where the part to be vulcanized is conveyed thru this chamber by an internal conveyor.

The internal oil is heated by a heater, circulated by the tunnel line and returned to the thermal heater, which is heated only with loss of energy, since there is no exchange of mass, just thermal exchange.

There are some hot air tunnels heated by electric resistance.

The speed of the products moving inside the tunnel and the speed for curing the compounds have to be adjusted in relation to the tunnel length.

In general, the vulcanization may be accelerated simply by increasing the temperature, when allowed by the shape thickness and the extruder speed because the new modified Organic Peroxides presented in this technology are not attacked by molecular oxygen.

The vulcanization temperature may be increased when working with elastomers that are highly resistant to the attack by oxygen, as in the case of EPDM. This compound may be vulcanized in hot air tunnel at a temperature over 250° C.

Generally, a water tank is located at the end of the tunnel line, where the shape receives a thermal shock immediately after leaving the very hot tunnel, as may be seen in FIG. 3. After the shock, the shape is conveyed for cutting, packing and then it is released for storage.

### Formulations

In this section, the inventor presents the formulations used for attesting the efficiency of the reticulation/cure of the modified Organic Peroxides, resistant to the presence de molecular oxygen, in the continuous vulcanization process in hot air tunnel.

Formulation (A) is a composition used for acceleration in the conventional system, via sulfur and accelerators, in hot air tunnel, according to Table 3.1 and formulation (B) is a composition used in the reticulation system based upon modified Organic Peroxide, resistant to oxygen, according to the following tables, both being conducted in Banburies with capacity of 43 liters,

TABLE E.6.1
EPDM formulation, accelerated with conventional
system (sulfur and donors).

| Conventional Formulation (A) | PHR | PHR |
|---|---|---|
| ETHYLENE-PROPYLENE-DIENE | 100 | |
| ZINC OXIDE | 1 | 10 |
| MINERAL CHARGES | 50 | 280 |
| PARAFFIN OIL | 40 | 240 |
| STEARIC ACID | 10 | 250 |
| POLYETHYLENE WAX | 1 | 5 |
| CALCIUM OXIDE | 1 | 5 |
| SULFUR | 3 | 15 |
| MBT | 0.3 | 2.5 |
| TMTD | 0.5 | 1.5 |
| ZBDC | 0.2 | 1.3 |
| MBT | 0.1 | 1 |
| DPG | 0.3 | 2 |

TABLE E.6.2
EPDM formulation, accelerated with Modified Organic Peroxide

| Formulation (B ) | PHR | PHR |
|---|---|---|
| ETHYLENE-PROPYLENE-DIENE | 100 | |
| ZINC OXIDE | 3 | 15 |
| CARBON BLACK | 30 | 280 |
| MINERAL CHARGES | 40 | 220 |
| PARAFFIN OIL | 10 | 120 |
| POLYETHYLENE WAX | 1 | 10 |
| DESICCANT or CALCIUM OXIDE | 3 | 15 |
| MODIFIED ORGANIC PEROXIDE | 3 | 20 |
| RETILOX/AIR | | |
| Other additives | 0.2 | 6 |

The formulas with modified Organic Peroxides resistant to oxygen are noticeably more compact, using much less ingredients.

Polymer Classification

The saturated or unsaturated polymers may be classified into plastomers and elastomers, the plastomers or plastics are subdivided into thermoplastics and thermofixed. The elastomers are polymers that may be repeatedly deformed at ambient temperature to at least twice their original length. Removing the stress, they should return to their original size. The thermoplastics are plastics with capacity to soften and flow when submitted to increased temperature and pressure. When they are removed from this process, they are solidified into products with defined shape.

New applications of temperature and pressure produce the same effect of softening and flowing. This change is a reversible physical transformation, thus they are recyclable. The thermofixed are plastics that soften once with heating, undergoing a cure process (irreversible chemical transformation) becoming hard. Posterior heating does not change their physical status. After the cure they become unmeltable and insoluble.

The used Polymers are Ethylene-Propylene-Diene (EPDM)

As we know, the history of the Ethylene-Propylene elastomers started with the discovery of a new class of catalysts based upon Aluminum-Vanadium discovered by the researcher Karl Ziegler.

A significant step for the rubber industry was the work of Giulio Natta, using the same class of catalysts obtaining a system able to produce amorphous Ethylene-Propylene copolymers with elastomeric characteristics.

The first large scale productions of Ethylene-Propylene copolymers for marketing in the rubber market started on the early 1960's, at that time the producers were the companies: Exxon, Enichem, E.I. Du Pont de Nemours and Uniroyal. During the next 20 years, several other producers installed their plants, exploring the constant growth of the market, which has been expanding until today.

The main characteristics that allow the interesting use of EPDM/EPM in the sectors; automotive, power cables and others, where the technical performance of the products versus price is a determinant factor; are the excellent properties of resistance to heat, aging, mechanical strength, resistance to ozone and oxidation and dielectric resistance.

The main molecular structure of the Ethylene-Propylene polymer of hydrocarbon origin presents completely saturated chains, i.e., no double bonds, which allows this rubber type to provide an excellent resistance to ozone.

The product also has excellent resistance to heat, oxidation, and polar fluids. The EPDM polymer presents a small residual unsaturation (double bonds), which is found peripherally to the main molecular principal chain and it is this residual unsaturation that allows the vulcanization with sulfur and accelerators.

These polymers may be blended (mixed) to other types of polymers that have already been described.

This patent application shall be directed to the market producing technical rubber shapes, commercially know as “Shapes” (“Perfil” in Brazil), as well as Electric Cables, Ductos and other already described products.

The Shape has may application areas such as, for example, automobile doors, between the carriage and doors, automobile trunks and windows, as glass sealing in civil construction, shapes used in bridges, among other varied applications.

The technical shape for this use complies with a very strict specification applied by the assembly industry, which shall use this shape, to the shape producing rubber industries, this being a product upon which the nature actions are extremely high, due to metropolitan pollution, acid rain, ozone and all the weathering caused by nature, in order to assure good quality for this product, the assembly industries impose strict specifications according to ASTM D 2000 standard.

Due to the attack of air oxygen and ozone, the specification requires using EPDM—Ethylene-Propylene-Diene (as third monomer), however, EPM—Ethylene Propylene Copolymer may be used, as well as a wide range of SATURATED and UNSATURATED polymers and their blends as already described.

The physical properties such as hardness, rupture stress and permanent deformation are established by an internal specification of the assembly company, or other industrial segments such as power cables, shapes for civil construction, etc. The most emphasized property is permanent deformation, heat resistance, since some shapes are used in bus doors and are submitted to constant compression.

The shape is a product with very large size, since it has to surround all the structure of the bus doors, this was a large problem for the rubber industry, since it was not economically feasible to use the pressing system for rubber reticulation, and in order to make the process economically feasible, the industry started to manufacture the shapes with a continuous vulcanization system, in hot air tunnel, with presence of oxygen.

However, this process presents many difficulties, one of these is the lack of ideal vulcanization of the shape when using the conventional vulcanization system, with large interference in the results, particularly the permanent deformation, obliging the assembly companies to accept products with deviations out of the specifications.

Charges

Carbon black is the most commonly used compound reinforcing charges used for production of black color products, even though, the mix of carbon black with mineral charges is also widely used by the vulcanized product industries, mineral charges such as: silica, kaolin (aluminum silicate), calcium carbonate, industrial talk, hydrated alumina, among others, are also commonly used in EPDM compounds.

Mineral charges are widely used in compounds for production of products in light colors, or together with carbon black, having cost reduction as basic function, however, they help in the processing capacity of the compounds, the industry also used a single mineral charge as white reinforcement, precipitated silica is the mostly used charge for EPDM compounds, when using sulfur as cure agent, the combination of silica with corn silane allows for vulcanized products with excellent mechanical properties.

In general, the mineral charges provide, to EPDM compounds (when compared with the properties provided by carbon black), low modules, high elongation, low resilience and high permanent deformation under compression, on the other side, we have easier processing, better electric isolation and lower cost for the compounds.

The two work formulations used two types of charges: carbon black (reinforcement charge) and Aluminum Silicate better known in the rubber industries as Kaolin (filling charge).

Plasticizer

The plasticizer for oil derived EPDM compounds is the most commonly used in EPDM compounds, paraffin and naphthenic oils are the types with better compatibility with the EPDM copolymer, for this reason these are the most widely used, the aromatic plasticizers are rarely used. The naphthenic plasticizers, even though presenting good compatibility with EPDM, are very volatile at high temperatures, requiring a careful selection for use. The volatility may be improved if the naphthenic oils are combined with paraffin oils in the composition, the paraffin plasticizers on the other side, are less volatile at high temperatures, both for processing and application of the vulcanized product, allowing the incorporation of high volumes to the compound, and also provide products with less permanent deformation under compression, which is one of the more frequent requests in the area of technical rubber shapes, this being the reason for using this type of plasticizer in base formulations (paraffin oil).

Process Aids

The process aids for Polymer compounds, such as EPDM, present easier processing, both for mixing the compound or shaping the products. However, as a precaution due to the large amount of charge included in the formulations, the PHR of polyethylene wax was added as process aids for improving the flow and surface finishing.

### ADVANTAGES

The advantages of this technology, via modified Organic Peroxides, resistant to oxygen, when using Reticulation/Cure of Polymers in the Continuous vulcanization process in Hot air tunnel, for production of shapes, are:
A) Incorporating high cure speed, increased productivity,
B) Increased process safety relative to the conventional acceleration via sulfur and accelerators, since the phenomenon of mass losses due to pre-vulcanization is avoided.
C) Improved physical properties
D) Reducing many ingredients in the formulation

The physical properties such as hardness, rupture stress and permanent deformation are established by an internal specification of the assembly company, or by other industrial segments such as power cables, shapes for civil construction, etc., the most emphasized property is the permanent deformation because the shapes are used in bus doors and are exposed to constant compression.

The other advantages obtained in the continuous vulcanization process in hot air tunnel in presence of oxygen due to the adoption of these new technologies are: increased productivity, Absence of Nitrosamines, (toxicity), allowing the use of polymeric blends, production of compact and spongeous products, colored products and recycling the waste of reticulates/cured products, reducing costs and protecting the environment.

The masses accelerated with these modified Organic Peroxides may be stored for months without occurring pre-cure, different from what occurs in the conventional vulcanization via sulfur and accelerators, where the mass may be subject to pre-vulcanization.

The Modified Organic Peroxides allow the reticulation/cure of saturated and unsaturated Polymers, with larger bonding force, in relation of any of the vulcanization systems via sulfur and accelerators, this fact allows more flexibility to the formulator and to those who specify the final product.

The modified Organic Peroxides resistant to oxygen grant a safe scorch, may be added to the mix in the Banbury (mass mixing equipment that may reach very high temperatures).

They improve the physical properties of the vulcanized product when compared with the vulcanization with Sulfur and accelerators, since this is much older, but has already arrived to the extreme ends of sophistication and improvement.

The invention of the Organic Peroxides resistant to oxygen, and the improvement of the continuous vulcanization process in hot air tunnel, object of the patent application, greatly improves this and other physical properties of the produced products, overcoming in quality, productivity and toxicity the current conventional vulcanization system, via sulfur and accelerators.

This innovative technology of modified Organic Peroxides, resistant to oxygen, presents a definitive solution to the tackiness phenomenon that used to occur on the surface of the parts, due to the attack by oxygen, in the products produced by the continuous vulcanization process, in hot air tunnel, when trying to use conventional Organic Peroxides; of dialkyl, dialkyl, peresther and perketal classes.

Comparison of Vulcanization/Polymeric Cure Systems

For the best demonstration of the invention/innovation we shall analyze and compare the Vulcanization and or reticulation/cure systems under industrial use.

Per definition, all the elastomers are giant polymeric macromolecules constituted by hydrocarbons that have mobility and movement when submitted to the action of a force.

During the reticulation/cure, these macromolecules are crosslinked one into another forming a huge macromolecular network with reduced mobility and movement.

The bonding is framed by crosslinking among the molecules.

This bonding is normally located between two carbon atoms of two different polymeric chains, sometimes without any atom or atoms between them and sometimes with one atom or atoms not necessarily of carbon.

Obviously, this is a reaction that occurs under heat and in the presence of a chemical agent that, as a consequence, leads to increasing molecular weight, resistance, hardness and stability of the polymer or compound containing the same.

Thermal Stability and Bonding Energy of Cure Systems

| | | SULFUR | NORMAL | MODIFIED |
|---|---|---|---|---|
| | SULFUR | DONORS | PEROXIDES | PEROXIDES |
| Crosslinking | S—S | C—S | C—C | C—R—C |
| Types | | | | |
| Bonding | 49 | 63 | 75 | 81 |
| energy | | | | |
| Kcal/Mol | | | | |
| Compression | 52 | 28 | 11 | 8 |
| Set (%) After | | | | |
| 70 hrs. at | | | | |
| 100° C. | | | | |

The cure system via Modified Organic Peroxides, resistant to oxygen, C—R—C, grants larger bonding force and better physical properties in relation to the conventional vulcanization via sulfur and accelerators.

Vulcanization System with Sulfur and Accelerators

The vulcanization process of rubber compounds, when submitted to high temperatures, under pressure, during a certain period, changes state passing from highly deformable plastic to elastic due to the vulcanization phenomenon.

For better understanding the physical-chemical effect of vulcanization it is necessary to imagine that the rubber macro-molecules in crude state present a type of cord curtain where the cords are hanging almost parallel one to another, without any bonding connecting them, however, after the effect of vulcanization, the macromolecules are reticulated forming a network of crossed links, as if the curtain cords were transformed into a giant fishing net, three-dimensional.

The vulcanization causes, due to sulfur or cure agents, the crosslink also called bonding, and normally between two or more carbon atoms belonging to different molecule chains.

The ingredients of the conventional vulcanization of rubber consist of the combination of the vulcanization activators that are used in the compounds with the objective of quickly activating the accelerators in order to increase the vulcanization speed of the compounds.

The activator systems most commonly used in the composition of conventional rubbers are the combinations of a metal oxide with a fat acid. Normally, the mostly used activator system is zinc oxide in contents from 3 to 5 PHR, and stearic acid, best known in the rubber industry as stearin, in a ratio of 1 to 3 PHR.

Less common, but also producing good results, is using other metal oxides such as; magnesium oxide, lead oxide, basic lead salts; and also oleic acids such as; lauric, palmitic acids, etc.

Basically, it is understood that the vulcanization activation occurs with the combination of zinc oxide with stearic acid originating zinc stearate, which is combined with the accelerator agents forming complex salts, which on their part, facilitate and accelerate the crosslinking of the rubber macro-molecules.

The vulcanization agents are ingredients added to the rubber compounds responsible for promoting the reticulations (crosslink) between the macro-molecules of the elastomers, during the vulcanization.

In order to transform the compound, initially with plastic characteristics, into elastic, such as those desired for the final products, the vulcanization agents may be classified into three categories as follows: sulfur, sulfur donors and non-sulfurous.

With a deeper analysis, we notice that the sulfur atoms react with the atoms of the double carbon olefilic bonds, as well as with the adjacent, forming the crosslinking (reticulations) between the elastomer molecules.

The sulfur that is more frequently used in rubber compounds is the soluble type, or also called rhombic sulfur, insoluble sulfur or amorphous sulfur are less frequently used due to being more expensive, however, this type of sulfur allows the compounds to maintain their surface adhesive (tack) characteristics for longer time, since it has no trend to outcropping. The sulfur contents as vulcanization agents in the rubber compounds may vary from 0.5 to 3.5 PHR, except when ebonite is desired, where the level may reach 30 PHR.

During the vulcanization of a rubber compound, sulfur may be combined in may ways to promote a huge reticulated network. We may find crosslink in the form of; monosulfides, disulfides, polysulfides, cyclic sulfides and cyclic polysulfides.

Depending on the sulfur content added to the compound, not all the sulfur atoms are combined with those of the elastomers, however, it is considered as satisfactory when it occurs as a minimum of one crosslink (reticulation) for each 180 units of monomer in the structural chain of vulcanized rubber.

The larger the reticulation of the macromolecular structure of a vulcanized compound and the smaller its mobility, it shall be more rigid, hard, and less flexible and when the structure is totally saturated with sulfur we obtain ebonite.

Sulfur donors are a determined type of vulcanization accelerator ingredients that contain sulfur in their constitutional structures. These ingredients are added to the rubber compounds and are decomposed releasing sulfur and then occurs the vulcanization of the rubber. Such ingredients are called “Sulfur Donors”.

When using a sulfur donor in the compounds, the elementary sulfur content may be reduced or even eliminated.

The compounds with low elementary sulfur contents are normally known as; compounds with “semi-efficient” cure system; and the compositions that do not use elementary sulfur, using only sulfur donors, are called “efficient” cure system.

The sulfur donors, during the act of vulcanization, release sulfur atoms to be combined with the atoms of the carbon chain of the rubber promoting the necessary reticulations for changing the compound status.

Products manufactured with conventional rubber compounds, using elementary sulfur, present low properties of resistance to heat and aging, this is due to the large number of polysulfidric bonds that occurs in the molecular chains of the elastomers.

If a large resistance to aging and heat is an important requirement of the product, the use of sulfur donor ingredients in duly metered ratios provides excellent results since the thermal stability of such ingredients is superior, besides providing compounds with smaller reversion trend, however, the compounds with “efficient” or “semi-efficient” cure system present reduction of the properties of dynamic fatigue resistance, maybe due to the smaller number of polysulfidric bonds of the vulcanized products.

Table 1F presents some organic sulfur donor accelerator ingredients, as well as the sulfur content that may be released during the vulcanization.

TABLE 1 F
List of the mostly used sulfur donors of the rubber industry.

| | | Sulfur |
|---|---|---|
| Commercial Name | Technical Name | content (%) |
| Sulfazan R | Dimorfoline Bisulfide | 31 |
| Tetrone A | Dipentamethylthiouran hexasulfide | 35 |
| TMTD | Tetramethylthiouran disulfide | 13 |
| CPB | Bibutylxantane disulfide | 21 |

Vulcanization accelerators are ingredients added to the rubber compounds with the main objective of significantly reducing the vulcanization time of the products, without harming the optimum required characteristics, on the contrary, improving even more the properties, particularly the resistance to aging of the products.

Regarding the cure speed promoted by the accelerators we have available the following types; slow action, intermediate action, semi-fast action, fast action with delayed starting, very fast and ultra-fast action.

Table 2 F lists the accelerators types with the functional classification and cure speed.

TABLE 2 F
list of the mostly used accelerators of the rubber industry.

| | COMMERCIAL | FUNCTION | |
|---|---|---|---|
| CHEMICAL GROUP | BRANDS | CLASSIFICATION | CURE SPEED |
| ALDEHYDE AMINES | HMT VULKACIT H | SECONDARY | FAST START WITH SLOW |
| | | | SEQUENCE |
| GUANIDINE | T.P.G. | SECONDARY | SLOW TO |
| | D.P.G. | | INTERMEDIATE |
| | D.O.T.G. | | |
| THIAZOLES | MBT | PRIMARY | SEMI-FAST |
| | MBTS | | |
| | ZMBT | | |
| SULFONAMIDES | VULKACIT AZ | PRIMARY | FAST WITH DELAYED |
| | TBBS | | STARTING |
| | VULKACIT CZ | | |
| THIOURANS | TMTM | SECONDARY | VERY FAST |
| | TMTD | | |
| | TETD | | |
| DITHIOCARBAMATES | ZDC | SECONDARY | ULTRA-FAST |
| | ZBDC | | |
| | ZEDC | | |

Reticulation/Cure System with Conventional Organic Peroxides and Progress Obtained with Modified Peroxides, Resistant to Oxygen.

The use of Organic Peroxides as agents for crosslinking was reported for the first time by Ostromislensk in 1915. One of the first peroxides to be developed for application as cure agent was dibenzoile peroxide, which at that time was used for meal treatment and that was used for vulcanization of natural rubber.

Until the middle 1950's, the industrial use of peroxides as crosslinking agents increased partially with the development of saturated rubbers, such as EPM and silicone rubber, which cannot be vulcanized with conventional sulfur cure system. As from this time, scientific and technical works have been conducted in this field; a good view of these publications appearing until 1957.

Organic Peroxides are used for reacting with elastomers containing saturated and molecular chains as well as unsaturated containing “double carbon bonds available”.

The hemolytic rupture of the Organic Peroxides into two oxy-radicals that may subtract hydrogen atoms from the polymeric chain, normally of tertiary carbons for forming polymeric radicals, and the combination of these radicals form a crosslink.

The characteristic of an Organic Peroxide is the peroxide group -0-0-, which by the hemolytic shearing may be decomposed to form two radicals.

The general formula for such compounds is R₁—O—O—R₂ where R₁ and R₂ symbolize radicals or an organic radical and one hydrogen atom. When both radicals R₁ and R₂ are hydrogen atoms, the simpler obtained form is H—O—O—H, i.e., hydrogen peroxide.

Peroxides are used because the generated carbon-carbon bonds are more stable than the sulfur-carbon bonds and as a result, a better resistance to heat aging is obtained.

Reticulation/Cure (Crosslinking)

The Organic Peroxides form free radicals that subtract hydrogen from the main chain of the polymer, originating polymeric radicals.

The combination of two radicals results in a reticulation (CROSSLINKING) with C—C bond forming the bonding energy

From the point of view of thermal stability, the reticulation with Organic Peroxides, since it has more bonding force, is much more stable than the carbon/sulfur/carbon bond and grant good properties relative to aging resistance.

Since the bonds are formed by the innovating system of reticulation/cure of the new Organic Peroxides resistant to oxygen, for application in Hot air tunnel, they have even stronger bonds as already described in the C—R—C bond.

Classification of Conventional Organic Peroxides

Dialkyl Peroxides have two organic radicals partially or totally aliphatic by nature, in this group of peroxides we find a class with some subgroups.

Dicumil Peroxide, of Dialkyl class, indicated for curing elastomers and plastomers and their blends, is a source of free radicals and is decomposed at a temperature of 179° C.

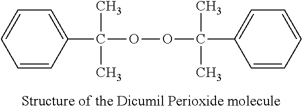

1,3 Di-(2-Tert.-Butyl Peroxide Isopropyl)Benzene is also a peroxide of the Dialkyl group, indicated for curing elastomers, plastomers and their blends, in the normal vulcanization processes and their best performance occurs when using a temperature over 180° C.

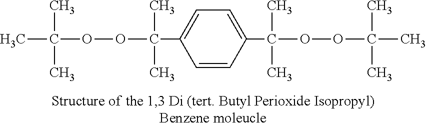

2,5-Dimethyl-2,5-di-(tert.-Butyl Peroxide)Hexane is an Organic Peroxide also from Dialkyl group, indicated for curing elastomers and plastomers and their blends, in the normal process of vulcanization and their best performance occurs at a temperature over 185° C.

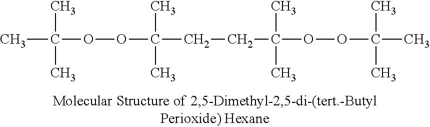

T. Butyl Perbenzoate from the class of aromatic Peroxyesters, where the acid hydrogen atoms were subtracted by an aliphatic radical, generally the ter-butyl radical, and their ideal reticulation/cure temperature is 175° C.

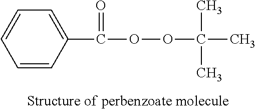

The diacyl peroxides, depending on the composition and the organic groups R₁ and R₂, may be subdivided into some subgroups, however, commercially, the most used is diacyl peroxide:

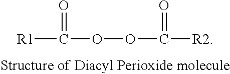

Diaroyl Peroxides, here the organic radicals constitute only aromatic groups.

The diaroyl peroxides include the bis(2,4-Dichlorobenzoil) and the first peroxide for crosslinking (Benzoil Peroxide) are more used in silicone rubber, as follows:

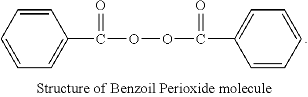

The 1,1-Di(Tert. Butyl Peroxide)3.3.5-Trimethyl Cyclohexane belong to peroxyketal class and may be considered as a derivate of the corresponding ketals, where the oxygen atoms and the ether bond were replaced by the peroxides groups and their commercial name is RETILOX TC:

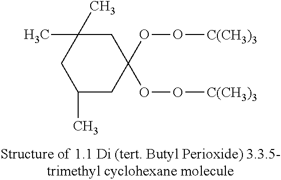

Innovation

In this invention, the main innovation is the molecular structural modification induced in the various classes of Organic Peroxides, described as follows, with the introduction of another radical in the molecular structure of the same, where also the Carbon-Carbon (C—C) bond is changed into Carbon-Radical-Carbon (C—R—C) creating a new range of modified Organic Peroxides, resistant to the presence de oxygen, for the reticulation/cure of polymers, in the continuous vulcanization process, in hot air tunnel, in presence of oxygen, without the occurrence of the cleavage/tacky phenomenon in the surface of the produced product.

The creation of modified Organic Peroxides used various types of Monomer Multifunctional Agents and their blends that significantly increased the number of bonds, protecting the Modified peroxides against the attack by Oxygen.

With this new series of modified Organic Peroxides, called RETILOX/AR series, we obtain a better performance even more in line with the state of the art required by the markets of automobiles, civil, energy, also allowing the production of white and colored products, without occurring cleavage on the surface of the products that are Reticulated/Cured by the process of continuous vulcanization in hot air tunnel.

### APPLICATION OF THIS INVENTION

By definition, all the elastomers are gigantic polymeric macromolecules, consisting of hydrocarbons that are provided with mobility and movement when submitted to the action of a force.

During the reticulation/cure, these macromolecules are interlocked one into another forming a huge macromolecular network with reduced mobility and movement.

The bond is formed by crosslinking between the molecules.

These bonds are normally located between two carbon atoms of two different polymeric chains, sometimes without any atom or atoms between them and other times with one atom or atoms not necessarily of carbon.

This is a reaction that occurs under heat and in the presence of one of the types of modified Organic Peroxide, resistant to oxygen, which, as a consequence, increases the molecular weight, resistance, hardness and stability of the polymer or compound containing the same.

Inhibition of the Crosslinking Reaction by Atmospheric Oxygen

During the reticulation/cure (crosslinking) by conventional Organic Peroxides, the reaction may be totally or partially inhibited, particularly on the surface, due to the admission of oxygen.

This effect is based upon the fact that the oxygen reacts extremely quickly with a substrate of the P* radicals; with the formation of POO* radical, this last one reacts comparatively slowly, resulting in no crosslinking occurring in these points.

The consequence of this is that the surface is inappropriately reticulated and in the case of elastomers, is tacky, i.e., cleaved obtaining the softening of the external surface of the shape, a phenomenon that does not occur with the modified Organic Peroxides described as follows;

Group 1—Diacyl Peroxide Class
1,1) Modified Benzoil Peroxide

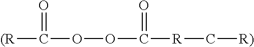

Commercial Name: RETILOX SI/AUTO-V
1,2) Bis(2,3-dichlorobenzoil)peroxide

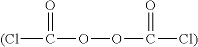

Commercial Name: RETILOX SI/AR

Group 2—Ester Peroxide Class
2,1) Modified T-Butyl Perbenzoate

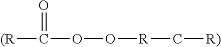

Commercial Name: RETILOX R 40/AR

Group 3—Peroxideketal (ketals) Class
3.1) Modified n-butyl-4,4 di(t-butylperoxide) Valerate
   Commercial Name: RETILOX BT/AR

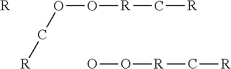

3.2) Modified 1,1-Di-(t-Butyl Peroxide)3,3,5-Trimethyl-Cyclohexane
   Commercial Name: RETILOX MT/AR

Group 4—Dialkyl Peroxide Class
4.1) Dicumil Peroxide
   Commercial Name: RETILOX HP 2006/AR

(R—O—O—R)—C—R—C
4.2) Modified Bis(Tert. Butyl Peroxide-Isopropyl)Benzene
   Commercial Name: RETILOX BIS 2007/AR
4.3) Modified 2,5-dimethyl-2,5-di-(peroxide t-butyl)Hexane
   Commercial Name: RETILOX DHBP/AR

### Formulation Indicating the Ingredients for EPDM/Modified Organic Peroxide.

| PHR | Ingredients | Type/data |
|---|---|---|
| 100 | EPDM or EPM or | Polymer |
| | Polymeric Blends | Ethylene Propylene Diene (terpolymer) |
| | | Ethylene Propylene (Copolymer) |
| 3-10 | ZINC OXIDE | Acceleration activator |
| 20-200 | CARBON BLACK | Reinforcing charge |
| 50-180 | CALCINED KAOLIN | Filling Mineral charge |
| 20-100 | PARAFFIN OIL | Paraffin plasticizer |
| 1-5 | POLYETHYLENE | Process aid. |
| | WAX | |
| 4-15 | ZINC OXIDE | desiccant. |
| 6-12 | RETILOX SERIES/ | Modified Organic Peroxide |
| | AR | |

### Test Standards

The Technical Standard aims to establish how to determine the characteristics, conditions or requirements of materials, products or equipment in accordance with the provisions of the specification standard. The physical properties after the vulcanization of the compound or product transform the same into a strong, elastic and insoluble material.

These characteristics are very important because they provide the performance of the product for the proposed service. The rupture strength or tenacity of a material is accessed by the load applied to the material per area unit at the rupture moment, elongation represents the percentage increase in the length of the part under tension, at the rupture moment, hardness is the strength opposed to the force of penetration of a spherical tip pin under a constant load.

The penetration value depends on the elastic module and the viscous-elastic behavior of the material under test. This value is converted into grades of hardness in Shore A, Shore D or IRHD (International Rubber Hardness Durometer) scale.

The permanent deformation under compression is the capacity of the compounds to retain their elastic properties after the extended action of compression, static or intermittent forces, it is the residual deformation presented by the used test sample after removing the compression load.

### Methods for Obtaining the Test Sample

For the process material, the test samples were prepared with two methods with the application of formula (A) and formula (B).

The first method was applied to the test sample prepared with the shape produced in the production, the other applied method was the test sample prepared with a plate used in the laboratory

The masses (A) and (B) were produced in Banbury, and submitted to physical tests in two different types of test samples, plate and shape.

### Procedure for Obtaining the Test Sample Cp (Shape)

The shape was produced following the sequences and process conditions as described in the following table,

TABLE 4.1
Process conditions for preparing test samples.

| Process Conditions | Formulation (a) | Formulation (b) |
|---|---|---|
| Mixing temperature (° C.) | 90-130 | 90-130 |
| Mixing time (min) | 12 | 8 |
| Banbury volume (kg) | 42 | 42 |
| Acceleration time in the cylinder | 7 | 4 |
| Extrusion temperature (° C.) | 35 | 35 |
| Thread diameter (mm) | 90 | 90 |
| Tunnel inlet temperature (° C.) | 150 | 150 |
| Tunnel outlet temperature (° C.) | 208 | 208 |
| Part temperature at the outlet (° C.) | 206 | 206 |
| Tunnel speed (m/s) | 4.5 | 4.5 |
| Thermal fluid temperature (° C.) | 216 | 216 |
| Tunnel length (m) | 32 | 32 |
| Cooling water temperature (° C.) | 20 | 20 |

### Scheme for Processing and Obtaining Test Samples

After the masses, formulation (A) and formulation (B), leave the Banbury, extrude the mass in the desired matrix, place the pre-formed in the hot air tunnel and after leaving, cut the shape using the dimensions provided in ASTM D 2240 standards.

Let the shape condition at the temperature of 23±2° C. and relative humidity of 50±5 for 24 hours and the test sample shall be well defined and free from imperfections that may affect the results.

### Procedure for Obtaining the Test Sample Cp (Plate)

After the masses, formulation (A) and formulation (B), leave the Banbury, mill with the cylinder, vertically mark, with the help of a pen, the milling at the end of the mass in the outlet direction of the cylinder, cut the mass with scissors, using the dimensions established in ASTM D 2240 standard, place the piece of cut mass with the marking arrow parallel to the higher part of the mold, place the mold in the press and remove the vulcanized part from the mold.

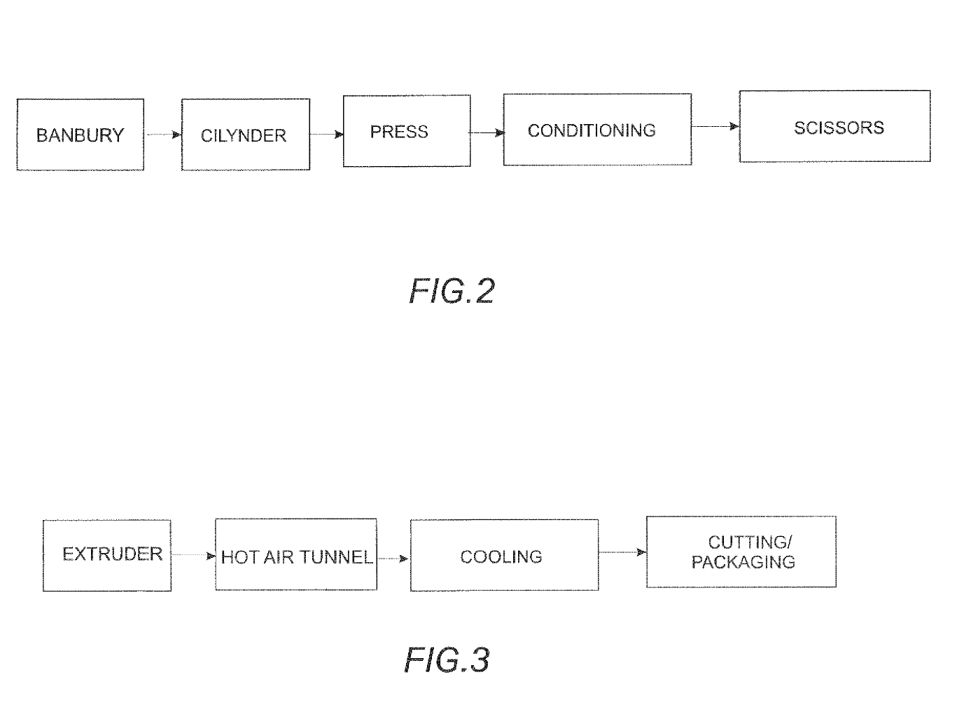

Let the place condition at a temperature of 23±2° C. and relative humidity of 50±5 for 24 hours and the test sample shall be well defined and free from imperfections that may interfere with the results. FIG. 2 shows the process scheme, and Table 4.2 presents the conditions for the same.

TABLE 4.2
Process conditions for preparing the test samples.

| Process conditions | Formulation (A) | Formulation (B) |
|---|---|---|
| Press temperature (° C.) | 180 | 180 |
| Press pressure (lb/in) | 14 | 14 |
| Pressing time (min) | 10 | 10 |

### Physical Tests

The physical tests with cut test samples were conducted according the standards, rupture strength (Tension) and elongation ASTM D 412, hardness ASTM D 2240, Permanent deformation under Compression ASTM D 395.

### Results

For determination of physical tests, 03 plate test samples were tested for each different test, since the shape test sample produces only the DPC. Table 4.3 presents the property data of the physical tests that are established by the assembly company to be obtained in the physical tests.

TABLE 4.3
Specification of physical properties
established by the assembly company

| Properties | Specification |
|---|---|
| Hardness (Shore “A”) | 70 ± 5 |
| Rupture strength (Mpa) | >7 |
| Elongation (%) | >200 |
| Permanent deformation under compression (%) | <25 |

Test Results (Shape)

Table 4.4 presents the results of the DPC test conducted on the shape.

TABLE 4.4
Data for Permanent Deformation under Compression (%).

| Number of | Formulation (A) | Formulation (B) |
|---|---|---|
| measurements | sulfur | modified peroxides |
| 1 | 39 | 19 |
| 2 | 38 | 18 |
| 3 | 39 | 17 |
| Median | 39 | 18 |

4.6.1 Test Result (Plate)

Table 4.5 presents the results of the tension test conducted on the laboratory plate.

TABLE 4.5
Data for rapture strength (tension) Mpa.

| Number of | | |
|---|---|---|
| measurements | Formulation (a) | Formulation (b) |
| 1 | 9.2 | 9.8 |
| 2 | 8.7 | 9.3 |
| 3 | 8.6 | 9.5 |
| Median | 8.7 | 9.5 |

Table 4.6 presents the results of the elongation test conducted on the laboratory plate.

TABLE 4.6
Data for Elongation (%).

| Number of | | |
|---|---|---|
| measurements | Formulation (a) | Formulation (b) |
| 1 | 400 | 360 |
| 2 | 490 | 390 |
| 3 | 450 | 400 |
| Median | 450 | 400 |

Table 4.7 presents the results of the hardness test conducted on the test sample of laboratory plate.

TABLE 4.7
Data for Hardness (Shore “A”).

| Number of | | |
|---|---|---|
| measurements | Formulation (A) | Formulation (B) |
| 1 | 74 | 75 |
| 2 | 75 | 73 |
| 3 | 74 | 73 |
| Median | 74 | 73 |

### CONCLUSION

The analysis of the results of the physical tests show a considerable improvement in the results for the tests of permanent deformation, tension, elongation and hardness in the formulation with peroxide in relation to sulfur, since with peroxides all the results complied with the standard established by the assembly company, on the contrary of sulfur, which demonstrated properties worse than peroxide, and did not complain with the standard required by the assembly company, since the DPC result did not reach the required by the standard.

We can see that the crosslink bonds formed by peroxide really demonstrated more efficiency in reticulation, when compared to vulcanization using sulfur, for this reason a considerable difference was obtained in the DPC test.

We may mention that previously the rubber area researchers observed the difference in the results of DPC for the crosslink reactions in relation to the conventional vulcanization, however, this phenomenon was observed in pressed, injected parts, this reaction was never tested in parts produced by the system of hot air tunnel due to the cleavage that occurred in the reticulated/cured parts when trying to use the conventional Peroxides in this application, due to atmospheric oxygen.

As from the invention and innovation of modified Organic Peroxides, resistant to oxygen, this problem was totally overcome, since the cleavage no longer occurs in the surface of the Shapes and the DPC tests may also be conducted in the reticulated part.

The importance of selecting each ingredient resulted in the success of the final results, using the correct EPDM and appropriate oil was fundamental.

The crosslink reaction did not occur in the tests conducted with amorphous EPDM and aromatic oil, making the part lifeless and tacky, i.e., with high level of cleavage, since these factors influenced the reaction of the modified Organic Peroxides.

With these fundamental points we obtained success in the real production of a shape with modified Organic Peroxides, resistant to oxygen, this being the large technological innovation of the moment.

Due to the obtained and mentioned results, we notice that the standard required by the assembly company requiring a result under 25% in the DPC test, the formulation that presented the best results was the one using modified Organic Peroxides, resistant to oxygen, in the composition, we may conclude that formulation (B) presented best results for DPC relative to formulation (A), and the other physical properties also obtained a superior result.

In relation to the produced shape, formulation (B) did not present any traces of cleavage and the visual aspect was accepted by the assembly company, we notice that the modified Organic Peroxide, resistant to Oxygen really inhibited the cleavage in the presence de oxygen.

Thus, the innovation was attested in laboratory and industrially, since the objectives were achieved, because it is possible to produce shapes and other important products, with excellent properties, in compliance with the modem standards and requirements of various important industrial segments.

Innovations

This patent application aims to demonstrate a new technology based upon the reticulation/cure, normally called vulcanization, with modified Organic Peroxides resistant to oxygen, which is a relevant innovation since it is able to inhibit the inconveniences caused by the presence of molecular oxygen, in compounds that were extruded and vulcanized in Hot air tunnel, in the presence of oxygen.

This invention also allows improving/optimization and larger production flexibility for the process of continuous vulcanization in air tunnel in presence of oxygen, and shall allow the production of electric cables, compact or hollow pipes, hoses, besides all the types of shapes, quickly and with better quality and safety.

The invention opens the way for use in the manufacture of extruded compounds with an innovation techniques as opposed to the traditional systems based upon the cure with sulfur, providing as advantages, the final characteristics of the products, such as improved mechanical properties, permanent deformation under compression and thermal aging, production of color products, better productivity, recycling the reticulated/cured products.

Until now, it was alleged that the peroxide cure could not be used for extruded parts with exposed surface during the production of atmospheric oxygen, requiring the use of techniques in more expensive equipment, among which we may mention the cure in autoclave, salt bath, fluid bed or hyperfrequency, etc.

The only exception in Organic Peroxide and very much adopted today, is using only dichlorobenzoil peroxide in the continuous vulcanization in hot air tunnel for silicone compounds, not allowing the used for other polymers.

The polymers and their blends reticulated/cured with modified Organic Peroxides resistant to oxygen present superior physical properties, particularly when compared to the vulcanized materials cured with sulfur and accelerators.

These properties provide large practical importance for the peroxide cure, mainly because the bonds are carbon to carbon or carbon radical carbon and not sulfur bridges as in conventional vulcanization.

1-3. (canceled)

4. Process for continuous vulcanization in hot air tunnel in presence of oxygen, characterized by including the following steps:
E.1—weighing of raw material in isolated environment;
E.2—mixing the same in Banbury type mixer;
E.3—acceleration of the process in an open mixer;
E.4—extrusion of the above mix;
E.5—continuous vulcanization in hot air tunnel at temperatures between 120° C. and 400° C.

5. A composition for reticulation, cure of polymers, saturated and unsaturated and their blends by the process of continuous vulcanization in hot air tunnel characterized by comprising:

| | | |
|---|---|---|
| Ethylene-propylene-diene | 100 | PHR |
| Zinc oxide | 3 to l5 | PHR |
| Carbon black 3 | 0 to 280 | PHR |
| Mineral charges | 40 to 220 | PHR |
| Paraffin oil | 10 to 120 | PHR |
| Polyethylene wax | l to 10 | PHR |
| Desiccant or calcium oxide | 3 to 15 | PHR |
| Modified organic peroxide Retilox/AR | 20 to 30 | PHR |
| Other additives | 0.2 to 6 | |

6. A modified organic peroxide including the introduction of another radical in the molecular structure of the conventional Organic Peroxide of C—C type, becoming a modified Organic Peroxide of C—R—C type with change in the carbon-carbon (C—C) bonding force, characterized by:
being resistant to the presence of oxygen for reticulation/cure of polymers;
being insensitive in the presence of carbon black;
may be processed by continuous vulcanization in hot air tunnel at temperatures between 120° C. and 300° C. under atmospheric pressure.

7. A modified organic peroxide in accordance with claim 1, characterized by being obtained from the conventional peroxides of the dialkyl, perester, perketal and dialkyl classes.
